(19) United States
(12) Patent Application Publication
(10) Pub. No.: US 2017/0043061 A1
(43) Pub. Date: Feb. 16, 2017

(54) Glycosaminoglycan Composition and Method of Use for Lubrication

(71) Applicant: ARTHRODYNAMIC TECHNOLOGIES, ANIMAL HEALTH DIVISION, INC., Lexington, KY (US)

(72) Inventor: Frank D. Marcum, Lexington, KY (US)

(21) Appl. No.: 15/307,468

(22) PCT Filed: May 4, 2015
(86) PCT No.: PCT/US2015/028985

§ 371 Date: Oct. 28, 2016

Related U.S. Application Data
(60) Provisional application No. 61/987,550, filed on May 2, 2014.

(51) Int. Cl. A61L 29/14; A61L 29/16; A61L 29/08

(52) U.S. Cl. CPC A61L 29/14; A61L 29/085; A61L 29/16; A61L 2400/10; A61L 2420/02; A61L 2300/232

(57) ABSTRACT

The invention provides lubricating compositions and methods of use thereof in man or in animals. Specifically, provided are lubricating compositions comprising effective amounts of chondroitin sulfate and hyaluronan, and optionally include N-acetyl D-glucosamine.

### CROSS-REFERENCE TO RELATED APPLICATIONS

This application claims priority to U.S. Provisional Application No. 61/987,550 filed May 2, 2014, the entire contents of which are hereby incorporated by reference.

### FIELD OF INVENTION

The invention is generally directed to a lubricating composition for various personal and medical usages. Specifically, the invention relates to a lubricating composition comprised of glycosaminoglycans and glycosaminoglycan precursors.

### BACKGROUND OF THE INVENTION

Lubricating products are known. Generally they comprise synthetic lubricating agents such as glycerine, propylene glycol, hydroxyethyl cellulose, etc. A variety of uses for these lubricating products include, e.g., skin care, bath and body care, deodorants, hand and foot care, facial care, hair care, shaving products, dental care, and personal lubrication. The ingredients in the formulated products in general serve as emollients, humectants, moisturizers, emulsifiers, lubricants, antimicrobials, cosmetics, fragrances, rheology modifiers, etc. Some of the products are solvent-based and others are water-based.

Most often lubricating products contain an active ingredient incorporated in a delivery vehicle. The major types of delivery vehicles most frequently fall into the following categories: (a) solutions; (b) emulsions, both oil-in-water and water-in-oil, including (for example) lotions and creams; (c) suspensions; (d) gels; and (e) solids (including semi-solids) including (for example) stick products. Nonvolatile hydrocarbons such as petrolatum, mineral oil, paraffin wax, ozokerite and the like have long been used in skin creams and lotions. These materials function as emollients by covering the skin with a hydrophobic occlusive film that prevents water loss from the skin surface to the environment. Likewise, animal fats and oils such as lanolin and its various derivatives, such as acetylated lanolins, have also been used in skin creams and lotions as emollients, depositing films on the skin that are hydrophobic, waxy and protective. Polymers such as polyethylene glycol (PEG), poly-1,2-propylene glycol (PPG), and block copolymers of ethylene oxide and propylene oxide, are now widely used in lubricating medical and personal care products.

Certain lubricating products may contain active ingredients and therapeutic agents for application to mucous membranes of the human body (the mucosa), potentially in combination with a medicament for treating the mucosa associated with certain diseases and disorders.

### SUMMARY OF THE INVENTION

A primary object of the invention is to provide a lubricating composition, and method of use thereof, adapted for a direct application to mucous membranes and other tissues of a human or animal body, particularly, the human or animal urinary system including kidneys, ureters, bladder, and urethra, to lubricate, prevent, alleviate or lessen, and treat affected mucosa areas associated with diseases and disorders in humans or in animals. The invention further contemplates any suitable usage of the inventive lubricating composition for any personal and medical lubricating, prevention, and treatment purposes. In particular, the invention provides methods of using the compositions comprising lubricating the outer surfaces of instruments and medical devices, such as a urinary catheter, to facilitate insertion, indwelling and removal from the body of an individual in need thereof.

In certain embodiments, the inventive lubricating composition comprises effective amounts of: chondroitin sulfate in combination with hyaluronan (hyaluronic acid), which may optionally be in solution and suspension with N-acetyl D-glucosamine. In certain embodiments, for example, the therapeutic amount of chondroitin sulfate can be from about 0.1 to 10 grams of chondroitin sulfate and the therapeutic amount of hyaluronic acid can be from about 10 mg to 1.0 gram per unit dose of the composition. In certain embodiments, the composition comprises about 10-250 mg/ml chondroitin sulfate and about 1-25 mg/ml hyaluronic acid. The chondroitin sulfate may preferably comprise a mixture of CS4 and CS6 chondroitin sulfate wherein the mixture can be from about 70% CS4 to about 30% CS4 and from about 30% CS6 to about 70% CS6. In addition, in some embodiments of the invention, the hyaluronic acid (HA) may be a Streptococcus derived (synthetically produced) HA having a molecular weight of at least about 250,000 Daltons and optionally may be at least about 500,000 Daltons. In other embodiments of the invention, the molecular weight of the HA of the invention is at least about 750,000 Daltons and optionally may be at greater than about 1,000,000 Daltons. The lubricating compositions of certain embodiments of the invention provide a chondroitin sulfate (as CS4 and CS6) that is in a combination with hyaluronic acid. The chondroitin sulfate of the compositions provided herein can be in solution or suspension with hyaluronic acid.

In certain embodiments of the invention, the lubricating compositions may further optionally include N-acetyl D-glucosamine. In certain embodiments, the hyaluronic acid and chondroitin sulfate of the lubricating composition is in a solution or suspension with N-acetyl D-glucosamine. The N-acetyl D-glucosamine provided in the lubricating compositions of the invention may provide a bridge to cross link with HA at its binding site as well as acting as a solution carrying precursor of the HA/CS link molecule versican/aggregan for the purpose of providing a supramolecular complex with link proteins to form a strongly hydrated space filling gel of poly-anionic glycosaminoglycan chains covalently attached to the core and contributing to the strength of GAG layers of the kidney, ureters, bladder, and urethra.

Compositions suitable for use in the present invention are described, for example, in U.S. Pat. No. 7,485,629 and U.S. Pat. No. 8,580,766, which are incorporated herein by reference in their entireties.

The invention provides that the lubricating compositions comprising therapeutic amounts of chondroitin sulfate in combination with hyaluronic acid, and optionally in combination with N-acetyl D-glucosamine, can be formulated to different suitable delivery vehicles, including, but not limited to, solutions, emulsions, lotions and creams, suspensions, gels, and semi-solids or solids. In certain embodiments, the lubricating composition of the invention lubricates the affected mucosa areas, as well as alleviates and reduces pain and inflammation or infections of the affected mucosa areas associated with certain physical and pathological conditions and diseases.

### DETAILED DESCRIPTION OF THE INVENTION

Additional objects, advantages and other novel features of the invention will be set forth in part in the description that follows and in part will become apparent to those skilled in the art upon examination of the foregoing or may be learned with the practice of the invention. Additionally, throughout this document, various publications and patents have been cited, the contents of which are incorporated herein by reference in their entirety.

Set forth in greater detail below are specific details related to lubricating compositions and methods of use thereof, adapted for an application to the surfaces of medical devices for insertion into a subject's body, such as a urinary catheter, or for direct application to mucous membranes of a human or animal body, particularly, the human or animal urinary system including kidneys, ureters, bladder, and urethra, to lubricate, prevent, alleviate or lessen, and/or treat affected mucosa areas associated with diseases and/or disorders. The invention further contemplates any suitable usage of the inventive lubricating composition for any personal and medical lubricating, prevention, and treatment purposes. The examples set forth herein are in no way intended to limit the scope of the invention. Those of skill in the art will realize that, given the teachings provided herein, many variations of the methods are possible that will fall within the scope of the invention.

In certain embodiments, the invention provides a lubricating composition comprising effective amounts of: chondroitin sulfate and hyaluronan (hyaluronic acid), and may optionally include N-acetyl D-glucosamine, in any suitable formulations, including but not limited to, solutions, emulsions, lotions and creams, suspensions, gels, and semi-solids or solids, to be applicable to any instrument, tissue, or mucous membranes of a human or animal body, particularly, the human or animal reproductive, digestive, and urinary systems, including kidneys, ureters, bladder, and urethra, to lubricate, prevent, alleviate or lessen, and treat affected mucosa areas associated with medical procedures, diseases and disorders in humans or in animals. One present embodiment of the invention provides a lubricating composition consists essentially of therapeutic amounts of chondroitin sulfate and hyaluronan, and optionally N-acetyl D-glucosamine.

While not wishing to be bound to any particular theory, it is believed that the glycosaminoglycans present in the lubricating compositions provided herein help to contribute to the return of homeostasis of mucosa and walls of human or animal organs, including but not limited to, kidney, bladder, ureter, and urethra, through the supramolecular complex of strongly hydrated space filling gel of poly-anionic GAG chains covalently attached to the core of the GAG layer in these organs, providing physical strength to these tissues. In addition to that effect, the incorporation of chondroitin sulfate into the compositions provided herein helps slow down the inflammatory process, by liganding to the CD44 and TSG-6 ligand receptor sites acting directly on the enzymes and inflammatory mediators that are released when inflammation is present.

The sodium hyaluronate (hyaluronan or hyaluronic acid) provided by the compositions serves to cover the surface or transitional epithelial lining/wall of these organs and mucosa tissues with a thin coating of the above supramolecule. Hyaluronan can also directly act as an inhibitor of inflammatory mediators by its direct effect on CD44 receptor ligands which mediates the migration of lymphocytes during inflammation. When present in the compositions of the invention, the N-acetyl D-glucosamine acts to link the supramolecular complex in the formulation as well as acting as a precursor to form new chains by the existing GAG layer.

The lubricating compositions of the invention provide a mixture comprised of the naturally occurring glycosaminoglycans: chondroitin sulfates CS4 and CS6, and hyaluronan (hyaluronic acid). Glycosaminoglycans are polysaccharides which occur widely in the animal kingdom. Glycosaminoglycans that are present in the tissues of vertebrate animals have mainly a linear structure which is repetition of a disaccharide units composed of two monosaccharides. Five kinds of glycosaminoglycans are found in the tissues and fluids of vertebrates: chondroitin sulfates, keratin sulfates, dermatin sulfates, heparin sulfates; hyaluronic acid and heparin.

Chondroitin sulfates are one component of certain embodiments of the lubricating compositions of the invention. In general, chondroitin sulfates are widely found in the connective tissues of animals in two forms of repeating disaccharides of D-glucouronic acid and N-acetyl galactosamine: CS4 sulfate where N-acetyl galactosamine holds an ester sulfate in its CS4 position or CS6 sulfate where the ester sulfate is in the CS6 position. Both CS4 and CS6 chondroitin sulfate function in the articular matrix as a major constituent. Chondroitin sulfates contribute to keep the intracellular matrix's normal characteristics through the binding with HA to form the core of the supramolecular complex, as well as slowing down the inflammatory process acting directly on the enzymes inhibiting the compliment cascade and by exhibiting anti-prostoglandin activity.

In particular, chondroitin sulfate is a long hydrophilic chain of repeating sugars. This glycosaminoglycan binds to proteoglycan molecules aiding in the attachment of the supramolecular complex to the core protein as well as the GAG tissue layer in the human or animal organs and mucosa tissues. Chondroitin in its sulfate form includes galactosamine, a primary substrate of hylauronan and a disaccharide pathway for proteoglycan synthesis secondary to the hexosamine pathways utilized for glycosaminoglycan production. Chondroitin stimulates the production of proteoglycans, glycosaminoglycans, and collagen, which are the building blocks of a healthy GAG layer of the certain human organs and mucosa tissues. Chondroitin sulfate also inhibits the secretion of degenerative enzymes by the liganding CS4 on the TSG-6 receptor responsible for inflammation. Chondroitin Sulfates are non-toxic and work synergistically with glucosamine to hydrate and repair the GAG layer of the certain human or animal organ walls and mucosa tissues. Chondroitin sulfate also works synergistically with hyaluronic acid to form the supramolecular matrix core to increase viscosity of the lubricating compositions of the invention and thereby increase the coating/protective properties of compositions as they bind to the GAG layer of walls and mucosa tissues of certain human or animal organs, such as kidney, bladder, ureter, and urethra. (see e.g., Hyaluroninin Synovial Joints: Molecular Seiving, Concentration Polarization & Secretion Regulation in vivo” Coleman et al., Matrix Biology Institute 2004).

Another component of certain embodiments of the lubricating compositions of the invention is hualuronic acid (HA) (also known as hyaluronan or sodium hyaluronate) which is a natural constituent of connective tissues and synovial fluid composed of repeating disaccharide units each consisting of D-glucoronic acid and N-acetyl D-glucosamine. Hyaluronan aids in providing nourishment and waste removal from the intracellular matrix. When combined with chondroitin sulfate, the exogenous hyaluronin present in the compositions of the invention acts synergistically with the chondroitin sulfate to lubricate, as well as to aid in the treatment and prevention of affected mucosa areas associated with certain disease and disorder conditions.

Therefore, one embodiment of the invention provides a lubricating composition adapted for an application to a medical device or for direct application to an affected and desired mucosa areas by providing an effective amount of the inventive lubricating composition to lubricate, alleviate or lessen at least one symptom associated with certain disease and disorder conditions in man or in animals, the lubricating composition comprising effective amounts of: chondroitin sulfate and hyaluronan in the substantial absence of other naturally occurring or synthetic glycosaminoglycans.

In general, the lubricating compositions of certain embodiments of the invention may optionally include N-acetyl D-glucosamine. N-acetyl D-glucosamine also possesses the ability to provide a bridge to cross link with HA at its binding site as well as acting as a solution for carrying precursors of the HA/CS link molecule versican/aggregan for the purpose of providing a supramolecular complex with link protein to form a strongly hydrated space filling gel of poly-anionic glycosaminoglycan chains covalently attached to the core and contributing to the strength of the GAG layer in the walls and mucosa tissues of certain human or animal organs, such as kidney, ureter, bladder, and urethra.

N-acetyl D-glucosamine is also a derivative of glucose obtained by chemical hydrolysis of chitin. This polysaccharide is readily soluble in water and extremely bioavailable. N-acetyl D-glucosamine binds to glucuronic acid as well as galactose making it a precursor to hyaluronic acid, keratan-sulfate and chondroitin sulfate. This unique derivative aids a proteoglycan, collagen and glycosaminoglycan production. N-acetyl D-glucosamine has also been shown to aid in the healing of soft tissue injury.

The embodiments of the invention are safe and non-toxic in the effective and therapeutic amounts as set forth herein. Each embodiment provides a specific benefit to lubricate the affected and desired mucosa areas, and to the treatment and prevention to thereby alleviate, lessen or prevent at least one symptom associated with certain physical and pathological condition in man or in animals. Thus, it can be realized that certain of the lubricating compositions of this invention, e.g., those comprised of chondroitin sulfate, N-acetyl D-glucosamine and hyaluronic acid provide a unique use of replacement components and metabolic precursors which can advantageously stimulate the production of glycosaminoglycans including hyaluronic acid, proteoglycans and collagen, thereby assisting the body's natural repair mechanisms and as a coating for the affected mucosa and epithelium tissues and walls of certain organs, such as kidney, ureter, bladder, and urethra, and to inhibit certain inflammatory mediators by its direct effect on CD44 and TSG-6, and Lyve receptor sites.

Another important aspect of the lubricating compositions of the invention is that they are especially well suited for use as a medical device for physical lavage or flushing of certain organs, such as the kidney, bladder, ureter, and urethra, as well as to provide a protective coating to the epithelium and mucosa. The highly negative ionic charge and unique characteristics of the lubricating compositions set forth herein act to directly trap or bind positively charged particles present in these tissues and organs, e.g., free radicals released from the inflammatory processes, and physically remove such particles from the epithelial surface of these organs and tissues. Because of their capacity for multidimensional disposition, hydrophilic nature, prominent presence of negative charges and lubricating/coating capabilities, the lubricating compositions provided herein also exhibit selective permeability, and support for the damaged epithelium and mucosa of certain organs and tissues of patients suffering from certain disease and disorder conditions, which require the return of the wall and mucosa (epithelium and interstitial matrix etc.) of affected organs and tissues to homeostasis.

Thus, in addition to the afore-mentioned active agents, it can be appreciated by one of skill in the art that the lubricating compositions of the invention can also comprise preservatives, pharmaceutically active carriers, excipients, stabilizers, buffers, antimicrobial growth inhibitors and the like and the use of such is contemplated by the invention.

It is contemplated by the invention that the lubricating compositions provided herein are sterile solutions and are suspensions comprised of chondroitin sulfate and hyaluronan, and optionally with N-acetyl D-glucosamine It is also contemplated that other formulations are possible and are within the scope of the invention, e.g., a powdered formulation suitable for reconstitution with suitable formulations, such as emulsions, lotions and creams, suspensions, and gels. In particular, it can be appreciated by one of skill in the art that the agents of the compositions can be stored in a freeze dried or lyophilized state for reconstitution and use at a desired time.

In certain embodiments, the invention comprises a lubricating composition comprised of chondroitin sulfate and hyaluronan, wherein the effective amount of chondroitin sulfate is from between about 0.1 to 10 grams of a suitable chondroitin sulfate per unit dose of the composition. In certain embodiments, the effective amount of chondroitin sulfate is about 10 mg/ml, 20 mg/ml, 30 mg/ml, 50 mg/ml, 100 mg/ml, 150 mg/ml, 200 mg/ml, and 250 mg/ml of the composition.

In one embodiment, the effective amount comprises about 1 gram of CS4 chondroitin sulfate, or about 1 gram of CS6 chondroitin sulfate or about 1 gram of a mixture of CS4 and CS6 chondroitin sulfate per unit dose. In some embodiments, the effective amount of chondroitin sulfate is about 1 gram of chondroitin sulfate comprised of about 40% CS4 chondroitin sulfate and about 60% CS6 chondroitin sulfate. In other embodiment, the chondroitin sulfate may comprise a mixture of CS4 and CS6 chondroitin sulfate wherein the mixture can be from about 70% CS4 to about 30% CS4 and from about 30% CS6 to about 70% CS6.

In certain embodiments, the effective amounts of hyaluronan include from about 10 mg to about 1.0 g of hyaluronan per unit dose of the composition. In some embodiments, the effective amount of hyaluronan is about 1 mg/ml, 2 mg/ml, 5 mg/ml, 10 mg/ml, 15 mg/ml, and 20 mg/ml of the composition.

It can be appreciated by one of skill in the art that the hyaluronan can be selected from among any of a number of commercially available sources. Likewise there are numerous commercially available sources of chondroitin sulfate, and N-acetyl D-glucosamine that are available for use in the compositions set forth herein.

In certain embodiments of the invention the lubricating compositions may optionally include effective amounts of N-acetyl D-glucosamine that are from about 0.5 grams to about 1.5 grams per unit dose of the composition. In some embodiments, the effective amount of N-acetyl D-glucosamine is about 1 gram per unit dose of the composition. In one embodiment, the CS and HA comprising the compositions of the invention are in about a 10% solution of N-acetyl D-glucosamine.

Another present embodiment of the invention provides a lubricating composition comprised of a sterile solution or suspension comprised of about 1 gram of chondroitin sulfate per 50 ml of composition (i.e., about 2% w/v or 200 mg/ml) as a mixture of about 40% CS4 and 60% CS6 chondroitin sulfate; and about 800 mg of hyaluronan (e.g., Na Hyaluronate) per 50 ml of the composition (i.e., about 1.6% w/v or 16 mg/ml). Still another embodiment of the invention provides lubricating compositions comprised of a 5 cc/unit dose wherein the lubricating composition comprises about 500 mg of a suitable chondroitin sulfate and about 25 mg of a suitable hyaluronic acid (e.g. about 500,000 Daltons) in a 10% solution of N-acetyl D-glucosamine. Yet another embodiment of the invention provides lubricating compositions comprised of a 10 cc/unit dose wherein the lubricating composition comprises about 1000 mg of a suitable chondroitin sulfate and about 50 mg of a suitable hyaluronic acid (e.g. about 500,000 Daltons) in a 10% solution of N-acetyl D-glucosamine. Making a final solution/composition comprising desired final concentrations of chondroitin sulfate and sodium hyaluronate in the composition is routine and well known in the art.

One example of the embodiment of the invention comprises method of using a 10 ml-50 ml unit dose of the lubricating composition. It can be appreciated that this unit dosage can be added to a suitable amount of a liquid, e.g., including but not limited to water, lactated ringers, normal saline, and DMSO, and other desirable delivery vehicles, such as emulsions, both oil-in-water and water-in-oil, including (for example) lotions and creams, suspensions, and gels. It can also be appreciated that more than one unit dose can be utilized per application and application regimen, and the dosage of certain usages can vary depending upon the severity of the condition, age and health of the patient and the like.

In certain embodiments the invention provides a lubricating composition comprising effective amounts of chondroitin sulfate and hyaluronan, wherein the molecular weight per unit dose of the composition is from between about 450,000 Daltons to about 1,100,000 Daltons. In some embodiments the invention provides a composition comprising effective amounts of chondroitin sulfate and hyaluronan, wherein the molecular weight per unit dose of the composition is from between about 500,000 Daltons to about 1,000,000 Daltons. In other embodiments the invention provides a composition comprising effective amounts of chondroitin sulfate and hyaluronan, wherein the molecular weight per unit dose of the composition is from between about 550,000 Daltons to about 700,000 Daltons, preferably about 600,000 Daltons.

In yet other embodiments the invention provides a lubricating composition comprising effective amounts of chondroitin sulfate and hyaluronan, wherein the molecular weight per unit dose of the composition is greater than about 450,000 Daltons. In some embodiments, the invention provides a lubricating composition comprising effective amounts of chondroitin sulfate and hyaluronan, wherein the molecular weight per unit dose of the composition is greater than about 550,000 Daltons.

In certain embodiments of the invention the lubricating compositions set forth herein can further comprise a therapeutic amount of a suitable antibiotic. Suitable antibiotics for use in the compositions provided herein include, but are not limited to any of the antibiotics that are known in the art for the treatment and prevention of infections and inflammations, and the like. As can be appreciated by one of skill in the art, the choice of antibiotic and therapeutic amount can depend upon many factors including, but not limited to, e.g., the etiology of the infectious organism being treated or personal preference of the treating veterinarian or physician.

The compositions of the invention can also further comprise any other therapeutic agents. Examples of other such therapeutic agents include, but are not limited to, synthetic and non-synthetic corticosteroid agents, nonsteroidal anti-inflammatory drugs, antirheumatics, immunoregulators, immunosuppressors, and interleukin production inhibitors. Specific examples of corticosteroid agents include, but are not limited to dexamethasone, hydrocortisone, triamcinolone, betamethasone, predonisolone, methylpredonisolone, halopredone, beclomethasone and the like.

Specific examples of non-steroidal anti-inflammatory agents include, but are not limited to diclofenac, indomethacin, ibuprofen, ketoprofen, aspirin, diflunisal, fulfenamic acid, floctafenine, tolfenamic acid, sulindac, fenbufen, salicylic acid, acemetacin, proglumetacin, nabumetone, protizinic acid, thiaprofen, oxaprozin, loxoprofen, alminoprofen, zaltoprofen, flurbiprofen, flurbiprofen and the like.

In certain embodiments, the lubricating compositions of the invention can further comprise of at least one pyrazolyl benzenesulfonamide compound, e.g., as set forth in U.S. Pat. No. 5,756,529 and U.S. Pat. No. 5,466,823, the contents of which are incorporated herein by reference. In particular, the lubricating compositions of the invention can further comprise a diaryl substituted pyrazole useful for treatment of inflammation and pain. It is specifically contemplated that the lubricating compositions of the invention can further comprise therapeutic amounts of any of the class of diaryl substituted pyrazoles their isomers, analogs and metabolites. In particular, these compounds reduce inflammation and pain primarily via inhibition of cyclooxygenase-2 (COX-2). In a preferred embodiment of the invention, the lubricating compositions provided further comprise a non-steroidal agent that reduces inflammation and pain primarily via inhibition of cyclooxygenase-2 (COX-2) and with the substantial absence of inhibition of cyclooxygenase-1 (COX-1). Examples of suitable diaryl substituted pyrazoles for use in the compositions of the invention include, but are not limited to, celecoxib, rofecoxib and the like.

Examples of other agents which may be added to the core compositions set forth herein include, axetil, piroxicam, tenoxicam, ampiroxicam, meloxicam, D-penicillamine, bucillamine, gold sodium thiomalate, auranofin, lobenzarit, salazosulfapyridine, methotrexate, cyclophosphamide, azathioprine, mizoribine, cyclosporin and the like.

In certain embodiments, the invention also provides a lubricating composition comprised of effective amounts of chondroitin sulfate; hyaluronan and a suitable antioxidant or free radical scavenger. In some embodiments, the lubricating compositions of the invention can further comprise an effective amount of suitable superoxide dismutase (SOD) or other antixoidant including, but not limited to, examples set forth in U.S. Pat. No. 6,127,356 to Crapo et al., the contents of which are incorporated herein by reference.

The foregoing descriptions of certain embodiments of the invention have been presented for purposes of illustration and description. They are not intended to be exhaustive or to limit the invention to the precise form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled.

What is claimed is:

1. A method of lubricating a medical device for insertion into the body of a subject or a mucosal area of a subject, comprising applying to the medical device or the mucosal area of the subject in need an effective amount of a lubricating composition comprising an effective amount of chondroitin sulfate and hyaluronan.

2. The method of claim 1, wherein said chondroitin sulfate is CS4, chondroitin sulfate, CS6 chondroitin sulfate, or a mixture of CS4 and CS6 chondroitin sulfate.

3. The method of claim 1, wherein said effective amount of chondroitin sulfate is between about 0.1 grams to about 10 grams per unit dose of the lubricating composition.

4. The method of claim 1, wherein said effective amount of hyaluronan is about 10 mg to about 1000 mg of hyaluronan per unit dose of the composition per unit dose of the lubricating composition.

5. The method of claim 1, wherein said composition further comprises an effective amount of N-acetyl D-glucosamine.

6. The method of claim 5, wherein said effective amount of N-acetyl D-glucosame is between about 0.5 grams to about 10 grams per unit dose of the lubricating composition.

7. The method of claim 1, wherein said lubricating composition further comprises one or more physically acceptable excipients.

8. The method of claim 1, wherein said lubricating composition is in a formulation selected from a solution, emulsion, lotion, cream, suspensions, gel, semi-solid, and solid.

9. The method of claim 1, wherein said lubricating composition is for topical application.

10. The method of claim 1, wherein said lubricating composition can also prevent or treat pain, inflammation, infection, or symptom associated with a disease or disorder condition.

11. The method of claim 1, wherein the medical device is a urinary catheter.
